(19) United States
(12) Patent Application Publication
(10) Pub. No.: US 2021/0150846 A1
(43) Pub. Date: May 20, 2021

(54) An apparatus for handling and delivering dose medication pouches

(71) Applicant: Newicon Oy, Kuopio (FI)

(72) Inventor: Ossi Parviainen, Kuopio (FI)

(73) Assignee: Newlcon Oy, Kuopio (FI)

(21) Appl. No.: 16/624,314

(22) PCT Filed: Jun. 22, 2017
(86) PCT No.: PCT/FI2017/050474

§ 371 Date: Dec. 19, 2019

(51) Int. Cl. G07F 17/00; G07F 11/16; G07F 11/62; G07F 11/64; B25J 15/00

(52) U.S. Cl. CPC G07F 17/0092; G07F 11/165; B25J 15/0028; G07F 11/64; G07F 11/62

(57) ABSTRACT

The invention relates to an apparatus for handling and dispensing dose medication pouches, where the dose medication pouches are stored in cassettes for dose medication pouches, and the apparatus includes: a control system for administration of data relating to storage location, drugs and use-by dates of the dose medication pouches, as well as for controlling functions of the apparatus; a drug cabinet including storage locations provided for the cassettes of dose medication pouches and extending through the drug cabinet from a filling side to a dispensing side; indicators on the filling side of the drug cabinet for controlling the filling of the cassettes of dose medication pouches; a collector on the dispensing side of the drug cabinet configured to collect the dose medication pouches from the cassettes of dose medication pouches placed in the storage locations according to the data relating to the storage location, the drugs and the use-by dates; and a dispensing station, to which, the collector is configured to transfer the dose medication pouches taken from the cassettes of dose medication pouches.

### FIELD OF THE INVENTION

The invention relates to an apparatus for handling and delivering dose medication pouches.

### BACKGROUND OF THE INVENTION

Dose medication pouches are used for the distribution of drugs to patients in such a way that the dose medication pouches are prepared manually or by machine at a pharmacy, from which they are delivered directly to patients or to wards in hospitals or other nursing institutions, to be administered to the patients by nursing staff. Normally, a single dose possibly containing one or more drugs is packed in a dose medication pouch to be administered at a given time. The dose medication pouches are stored in the form of rolled up bands of dose medication pouches, a number of dose medication pouches being attached to each other. It is thus easy to prepare several doses of drugs, for example the medication for two weeks, on a single roll of dose medication pouches.

There is a specific filling device for filling dose medication pouches, although the filling can still be performed manually as well. In connection with the filling, the dose medication pouches are equipped with a label or the like, on which the data of the drugs packed in the pouch is printed in text format and/or as a bar code or a matrix bar code. The dose medication pouches filled by the device for filling dose medication pouches are then stored in a drug cabinet, from which the nurse responsible for the medication of the patients detaches a dose medication pouch when starting to administer drugs. Thus, the dose medication pouches are presently collected manually in such a way that the nurse collects the dose medication pouches to be administered to the patient at said time of administration, from the storage of dose medication pouches (e.g. a drug cabinet), and delivers the dose medication pouches to the patient. Naturally, drugs can be delivered to several patients on a single round. A disadvantage in this routine of prior art is the manual collecting of dose medication pouches, involving a lot of work in hospitals and nursing homes. Another disadvantage in the present method is the risk of mistakes, because it is possible that the nurse picks up a wrong dose medication pouch from the drug storage, or administers a dose medication pouch, correctly selected as such, to a wrong patient, or fails to notice that a drug in a dose medication pouch is outdated.

### BRIEF SUMMARY OF THE INVENTION

It is an aim of the invention to provide a novel apparatus for the dispensing of dose medication pouches to facilitate the handling and dispensing of dose medication pouches, to prevent mistakes in the administration of dose medication pouches, and to make sure that the drugs dispensed in the dose medication pouches are not outdated.

The aim of the invention is achieved by an apparatus comprising a drug cabinet having storage locations provided for cassettes of dose medication pouches and extending from the first side to the second side of the drug cabinet, the first side being provided with photo controlled filling openings for the storage locations, and the second side being equipped with an automated collecting device for collecting dose medication pouches from the cassettes of dose medication pouches placed in the drug cabinet, as well as a dispensing station to which the collecting device is configured to transfer the dose medication pouches ordered by the user and collected from the cassettes of dose medication pouches. To put it more precisely, the apparatus according to the invention is characterized by what is presented in the independent claim 1. Dependent claims 2 to 16 present some advantageous embodiments of the apparatus according to the invention.

The apparatus according to the invention has the advantage that the manual collecting of dose medication pouches from rolls of dose medication pouches placed in the drug cabinet is automated, whereby the risk of human error in the dispensing of dose medication pouches is reduced, because the filling of the drug cabinet with dose medication pouches is performed in a photo controlled manner, and because the collecting of dose medication pouches upon picking them up from the drug cabinet is performed by an automated collecting device which collects the dose medication pouches from the drug cabinet on the basis of storage location data in the control system of the drug cabinet. Furthermore, the apparatus according to the invention has the advantage of speeding up and facilitating the handling of dose medication pouches because the manual handling and removal of dose medication pouches from rolls of drug medication pouches is reduced.

### DESCRIPTION OF THE DRAWINGS

In the following, the invention will be described in more detail with reference to the appended drawings, in which

FIG. 1 shows an apparatus according to the invention in a diagonal view from the front;

FIG. 2 shows the apparatus of FIG. 1 in a diagonal view from the side;

FIG. 3 shows an arm of an industrial robot belonging to the apparatus according to the preceding figures, as well as a device for handling dose medication pouches, placed at the end of the arm, in a more detailed view;

FIG. 4 shows a cassette for dose medication pouches, for use in the apparatus according to the preceding figures; and

FIG. 5 shows dose medication pouches to be handled and dispensed by the device according to the preceding figures, the pouches being still attached to each other in a rolled-up band of dose medication pouches.

### A DETAILED DESCRIPTION OF PREFERRED EMBODIMENTS OF THE INVENTION

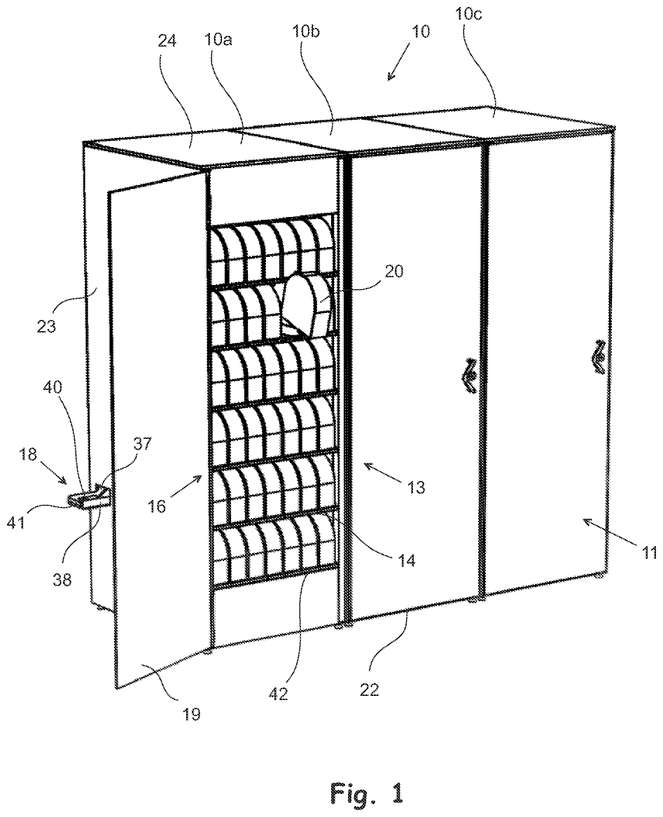

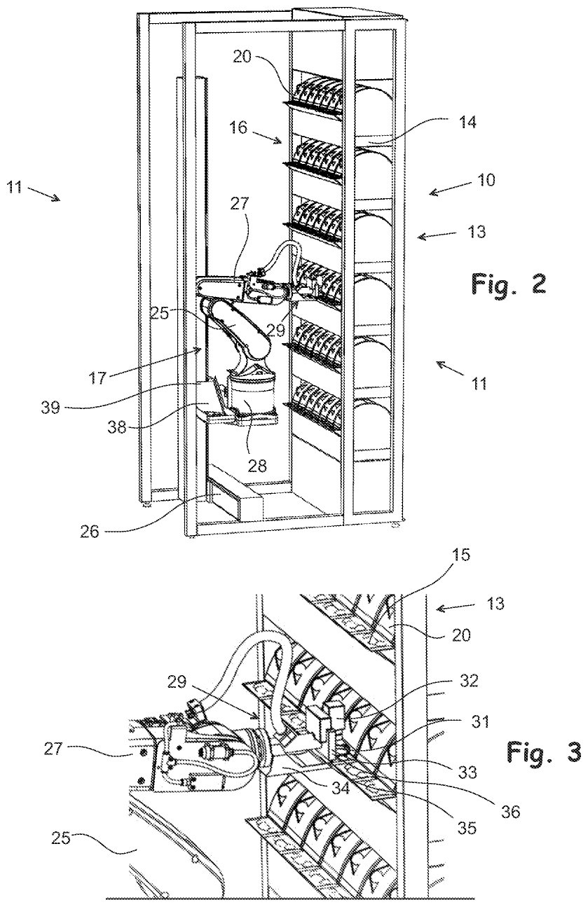

The apparatus shown in FIGS. 1 to 3 comprises a drug cabinet 10 having a filling side 11 and a dispensing side 12. On the filling side 11, there is a rack 13 with shelves 14 comprising storage locations 16 which extend from the filling side 11 to the dispensing side 12 of the drug cabinet 10. On the dispensing side 12 of the drug cabinet, a collector 17 and a dispensing station 18 are provided, to which dispensing station the collector 17 is configured to transfer dose medication pouches 15, provided in bands 30 of dose medication pouches stored in cassettes 20 for dose medication pouches, from the storage locations 16 of the rack 13 on the dispensing side 12 of the drug cabinet 10. In this embodiment, the apparatus also comprises indicators 42 placed at the edge of the racks 14 on the filling side 11 of the rack of the drug cabinet 10, for guiding the filling of the storage locations 16 in the drug cabinet 10, as well as doors 19 lockable by electronic locks to prevent the removal of cassettes 20 of dose medication pouches from the filling side 11 and possible unauthorized use of drugs contained in the dose medication pouches 15. Furthermore, the apparatus also comprises a control system for controlling the apparatus and the handling and dispensing of dose medication pouches 15 by it. The control system comprises a central processing unit with a main storage and a storage space (e.g. an SSD hard disk), a display, a keyboard. Moreover, the control system may comprise a bar code/matrix bar code reader for identifying dose medication pouches and/or for reading data relating to the drugs contained in them into the memory of the control system of the apparatus.

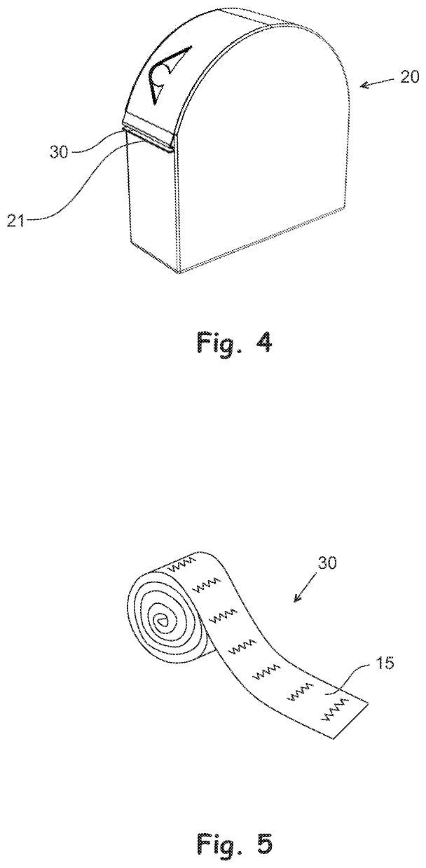

An example of dose medication pouches 15 to be handled by the apparatus is shown in FIG. 5. In the band 30 of dose medication pouches rolled up, the dose medication pouches 15 are attached to each other, one after the other, so that a single dose medication pouch 15 can be detached from the preceding dose medication pouch 15 by cutting off the band of dose medication pouches between two successive dose medication pouches. The cutting point may be marked to facilitate the selection of a suitable cutting line. The bands 30 of dose medication pouches have such a length that when rolled up, they can be fitted in a cassette 20 for dose medication pouches shown in FIG. 4. The cassettes 20 for dose medication pouches are equipped with a dispensing slot 21 through which the band of dose medication pouches may be pulled out of the cassette for dose medication pouches.

The drug cabinet 10 is, in this case, composed of three identical modules 10a to 10c. However, the number of modules 10 to 10c placed next to each other may vary in embodiments of this kind; that is, it may be one or more. Consequently, the number of modules placed next to each other defines the size (width) of the apparatus and thereby the number of cassettes 20 of dose medication pouches to be stored in it. Each module 10a to 10c comprises a frame 21 as well as a lower wall 22, side walls 23 and an upper wall 24 mounted on the frame. When two or more such modules 10a to 10c are mounted next to each other, the side walls opposing each other may be eliminated, if desired, in which case the modules constitute a single uniform drug cabinet 10. The width of the modules 10a to 10c is designed to accommodate a given number of cassettes 20 (in this case 7 cassettes) of dose medication pouches on the shelves 14 between the side walls 23 of the drug cabinet 10. The depth of the rack 13 of the drug cabinet 10, in turn, is defined so that the cassettes 20 for dose medication pouches to be used in the apparatus extend from the edge of the filling side 11 to the edge on the dispensing side 12 of the shelves 14 in the rack 13.

The storage locations 16 are arranged on the shelves 14 of the rack 13 in such a way that the site of a storage location 16 is determined by the adjacent cassettes 20 of dose medication pouches. As mentioned above, the width of the modules 10a to 10c is defined so that a given number of adjacent cassettes 20 of dose medication pouches fit between their side walls 23. Because the cassettes 20 of dose medication pouches are equal in width, a cassette 20 placed in a given storage location 16 on a given shelf 14 in the rack is always placed at a given site. Therefore, the sites for retrieving dose medication pouches 15 may be programmed for the collector 17, the sites always corresponding to the same storage locations 16 for cassettes 20 of dose medication pouches in the rack 13. However, it is possible to provide the storage locations 16 with e.g. intermediate walls, guide grooves, protrusions, or corresponding spacers for storage locations, fixed between the storage locations 16 on the shelves; but because the cassettes 20 of dose medication pouches are equal in width and are always stored primarily in the drug cabinet 10, they remain sufficiently precisely in the storage locations 16 allocated for them in the rack 13 of the drug cabinet 10 even without such additional elements. Nevertheless, if the shelves 14 are kept partly empty, the intermediate walls, guide grooves, protrusions or the like secure that the cassettes for dose medication pouches remain in place in the lateral direction.

The collector 17 on the dispensing side 12 of the drug cabinet 10 comprises an industrial robot 25 with an arm, and guide tracks 26 mounted on the module frame 21. The industrial robot 25 is configured to move along the guide tracks in the width and height directions of the drug cabinet 10 in such a way that its arm 27 reaches to retrieve dose medication pouches 15 from any storage location 16 in the rack 13.

The function of the collector 17, in this case the industrial robot 25, is to retrieve the dose medication pouches 15 ordered by the user of the apparatus from the storage locations 16 in the rack 13, and to transfer them to the dispensing station 18 by the side of the apparatus. The positions of all the storage locations 16 in the rack 13, as well as the dose medication pouches 15 contained in them and the drug dispensing data of the drug doses contained in them, are stored in the control system of the apparatus. The control system may thus control the collector 17 to retrieve a dose medication pouch 15 containing the ordered drug from the storage location 16 in which it was placed when the drug cabinet 10 of the apparatus was filled.

To enable the movement of the industrial robot 25 along the guide tracks 26, the industrial robot 25 is mounted on a transfer frame 28 suitable for supporting it and movably mounted on guide tracks. For collecting dose medication pouches 15, the transfer frame 28 is moved by suitable transmission devices (e.g. a cogged belt and cogged belt pulleys) powered by actuators (e.g. stepping motors), along the guide tracks 26 to a desired position with respect to the rack 13 of the drug cabinet 10. The movements of the transfer frame 28 may be configured to be controlled in such a way that the transfer frame 28 is always moved to a position which is as suitable as possible with respect to the storage location 16 of the rack 13 from which dose medication pouches 15 are intended to be retrieved, taking into account the reach of the arm 27 of the industrial robot 25.

A manipulator 29 for dose medication pouches 15 is provided at the end of the arm 27 of the industrial robot 25. The manipulator 29 comprises a pincher roll 31 which the manipulator 29 may rotate in different directions and which the manipulator 29 may press against the leading end of the dose medication pouch 15, pulled out of the cassette 20 of dose medication pouches and placed against the edge of the shelves 14 on the dispensing side 12 of the rack 13. Thanks to these functions, the manipulator 29 may grip the band 30 of dose medication pouches pulled out of the cassette 20 of dose medication pouches, and uncoil it from the cassette 20 for taking dose medication pouches 15 from the cassette 20. Furthermore, the manipulator 29 comprises a positioning sensor 32 operated by the control system for determining when the manipulator 29 has pulled out one dose medication pouch 15 from the band 30 of several successive dose medication pouches contained in the cassette 20 of dose medication pouches, as well as a cutter 33 by which the manipulator may cut the band of dose medication pouches pulled out of the cassette 20, at the joint between two successive dose medication pouches 15. For the dose medication pouch 15 pulled out and detached from the rest of the band 30 of dose medication pouches, the manipulator comprises a transfer grip 34 in which the dose medication pouch 15 is left after it has been pulled out of the cassette 20 of dose medication pouches and cut off the band 30 of dose medication pouches pulled out of the cassette 20. Furthermore, the manipulator 29 may comprise a labelling device 35 and/or a printer 36 by which the manipulator may print out data (e.g. patient's name and data on the drugs contained in the dose medication pouch 15) for the dose medication pouch 15 taken from the cassette 20 before it transfers the retrieved dose medication pouch 15 to the dispensing station 18. The printer 36 may be e.g. an ink jet printer or another printer suitable for the purpose.

The transfer to the dispensing station is performed by moving the arm and the transfer frame 28 of the industrial robot along the guide tracks 26 so that the arm 27 of the industrial robot 25 reaches the outlet 37 of the dispensing station 18.

In this embodiment, the dispensing station 18 is a discharge chute 38 placed in an outlet 37 on the side wall 23 of the module 10a on the left hand side of the drug cabinet 10 and extending from the inside to the outside of the drug cabinet 10. The part of the discharge chute 38 placed inside the drug cabinet is in an inclined position so that when a dose medication pouch 15 is placed in a feed point 39 inside, it will slide by gravity to a dispensing point 40 outside the drug cabinet. The dispensing point 40 of the discharge chute 38 outside the drug cabinet is provided with a flat surface on which all the dose medication pouches 15 sliding along the discharge chute 38 stop. When bringing dose medication pouches 15 to the feed point, the manipulator tilts the transfer grip 34 and moves the pincher roll 31 so that the dose medication pouch 15 in the transfer grip 34 is enabled to slide by gravity to the feed point of the discharge chute 38. The dispensing point 40 at the end of the discharge chute 38 is provided with a vertical end wall 41 which stops all the dose medication pouches 38 entered in the discharge chute 38 at the same point of the dispensing point 40. In this way, all the dose medication pouches 15 entered in the discharge chute 38 are brought to a bundle in the same order as they were entered in the discharge chute 38. The dispensing point 40 may also be equipped with a recess having the size of a dose medication pouch 15, whereby several dose medication pouches 15 fit on top of each other at the dispensing point 40, and the dose medication pouches 15 remain more securely on top of each other in the same order as they were entered in the discharge chute 38.

On the filling side 11 of the drug cabinet 10, the shelves 14 of the rack 13 are covered by doors 19. In this embodiment, the edges of the shelves are equipped with indicators 42 at each storage location on each shelf 14. The indicators 42 are indicator lights (e.g. LEDs) controlled by the control system controlling the apparatus. The function of the indicators is to control the filling of the cassettes 20 for dose medication pouches into the storage locations 16 of the rack 13 when the drug cabinet 10 is being filled. Thus, the indicators 42 operate in such a way that the control system uses them to indicate the storage location 16 in which a cassette 20 of dose medication pouches to be inserted into the apparatus shall be placed. The indicators 42 may also be used to indicate that a cassette 20 of dose medication pouches in a storage location 16 of the rack 13 has become empty and should be filled with a new (rolled-up) band 30 of dose medication pouches. The indicators 42 may thus operate e.g. in such a way that a flashing or e.g. red light indicates that the cassette 20 of dose medication pouches in said storage location 16 is empty.

On the filling side 11 of the drug cabinet 10, the shelves 14 of the rack 13 are open at front when the door 19 of said module 10a to 10c is open. In the embodiment shown in FIGS. 1 to 3, the doors 19 are made of glass or another transparent sheet material whereby the indicators 42 at the front edges of the shelves 14 of the rack 13 are visible through the doors. However, the doors might also be made of an invisible sheet material (e.g. metal) in the same way as the lower wall 22, the side walls 23 and the upper wall 24. In this case, each door 19 would comprise at least one indicator for indicating the module 10a to 10c comprising a storage location 16, in which there is a need to refill or replace a cassette 20 of dose medication pouches.

In the drug cabinet 10 according to the apparatus shown in FIGS. 1 to 3, cassettes 20 of dose medication pouches are placed in storage locations 16 in the rack 13 under control of the control system of the apparatus. Before this, the dose medication pouches 15 in the bands 30 of dose medication pouches in the cassettes 20, as shown in FIG. 4, are filled with patient-specific drug doses according to the drug doses intended to be dispensed by the apparatus. One cassette 20 of dose medication pouches may contain, for example, the drugs for a single patient for several days in such a way that one dose medication pouch 15 contains the drugs for one time of administration. The dose medication pouches 15 may be filled by e.g. a specific device for filling dose medication pouches 15, forming a labelled band 30 of dose medication pouches of two band-like plastic strips, one of them having an outer surface formed by a finished paper surface on which data relating to the bagged drugs, bar codes or matrix bar codes (i.e. for example QR codes) may be simultaneously printed out by the filling device. Next, the bands 30 of dose medication pouches containing drug doses are inserted in the cassettes 20 of dose medication pouches in such a way that the first dose medication pouch 15 is inserted through the outlet at the front end of the cassette 20, extending partly outside the cassette 20 of dose medication pouches. After this, the drug cabinet 10 of the apparatus is filled with the cassettes 20 of dose medication pouches, one by one, in such a way that the data of each cassette 20 of dose medication pouches is first read by a bar code/QR code reader connected to the control system of the apparatus. Alternatively, the data can be entered in the control system manually via a keyboard belonging to the apparatus. At the filling stage, after the reading/entering of the data on the cassette 20 of dose medication pouches, the control system controls, with aid of the indication means 42 at the edges of the shelves in the rack 13 of the drug cabinet 10, to which storage location 16, of which module 10a to 10c, and of which shelf 14, the cassette 20 of dose medication pouches shall be placed. In this way, the control system maintains information on the location of said cassette 20 of dose medication pouches filled in the drug cabinet 10 of the apparatus.

When dose medication pouches are dispensed by the apparatus of FIGS. 1 and 3, the apparatus may operate e.g. in the following way.
A user logs in the control system of the apparatus via a user interface in the control system. In this context, the control system makes sure that the user has the right to dispense drugs from the apparatus.
After logging in the system, the user enters the data of one or more patients in the control system, for dispensing their drugs by the apparatus.
After the login and the entry of the data of the patient/s, the user defines e.g. the number of drug doses or a period of time for which the drugs are to be dispensed.
Next, the control system starts to operate the collector 17 of the apparatus and displays the patient whose dose medication pouches 15 are to be transferred from the storage locations 16 of the apparatus into the discharge chute 38 of the apparatus, and requests the user for permission to start.
If the user accepts the transfer of the proposed dose medication pouches, the collector 17 starts to transfer dose medication pouches 15 intended for the proposed patient into the discharge chute 38 of the apparatus.
After all the dose medication pouches 15 of said patient have been transferred to the discharge chute 38 of the apparatus, the control system indicates that the medication of the patient is ready and requests the dose medication pouches intended to be administered to the patient to be picked up from the dispensing point 40 of the discharge chute 38.
Having done this, the user acknowledges (e.g. by pressing the “enter” key of the keyboard) that the dose medication pouches 15 intended for said patient have been removed from the dispensing point 40 of the discharge chute 38.
Next, the apparatus asks if further dose medication pouches 15 are to be dispensed for other patients, or in case several patients were initially selected, the apparatus asks if the drugs for the next patient in the list of selected patients are to be dispensed next.
After all the dose medication pouches 15 to be dispensed have been dispensed, a negative reply is given to the previous question, whereby the apparatus stops its operation and e.g. asks if a report on the drugs dispensed at this time of usage is to be stored/printed out.

In many cases, there are several dose medication pouches 15 to be dispensed for a patient at a time. Therefore, it is advisable to bundle the dose medication pouches 15 to a single bundle, e.g. by a staple, a bundling ring or another suitable way. This can be done by a bundling device placed outside the apparatus and mounted close to the dispensing point 40 of the discharge chute 38, for example by a stapler or a punch and bundling rings, to which the dose medication pouches dispensed for a given patient are threaded in such a way that they are reliably held in one bundle.

The dispensed dose medication pouches 15 may be delivered to the patients in the conventional way manually so that a nurse brings the bundle of dose medication pouches 15 to the patient whose name was printed out on the printing section of the dose medication pouches 15 by the manipulator or the bagging device of the apparatus. Alternatively, e.g. a delivery cart may be used which is equipped with a compartment for each patient in the respective ward or room, in which compartment the bundle of dose medication pouches may be placed and from which the dose medication pouches 15 may be delivered in the order according to the prescriptions of the patient. The delivery cart may also be implemented in such a way that it comprises, in addition to or instead of compartments in a chest of drawers, one or more dispensing trays with dispensing units, in which the nurse responsible for the administration of drugs discharges the drug doses contained in the dose medication pouches, before taking the delivery cart to the location of administration of the drugs. For implementing the dispensing of drugs in this reliable and safe way, the drug dispensing tray may be, for example, a drug dispensing tray according to an earlier patent application by the applicant, as presented in WO 2013/079792, equipped with an indicating device connectible to a portable computer or another data processing device.

The apparatus according to the invention may be implemented, in many respects deviating from the example embodiment presented above. For example, the collector belonging to the apparatus does not necessarily have to be based on an industrial robot with an arm but may also be a different device moving the manipulator in a corresponding way according to control commands by the control system. For example, in the case of a collector movable along guide tracks, the guide tracks of the collector may be placed so close to the shelves of the rack on the dispensing side of the shelves of the drug cabinet, that the manipulator can be steered to the end of the band of dose medication pouch at each storage location merely by means of the transfer frame and a straight bar movable back and forth towards and away from the edges on the dispensing side of the shelves of the rack by means of a motor. In this case, however, the discharge chute has to be such that the collector having a more limited movement than the industrial robot (i.e. a movement in only one direction) is capable of bringing the dose medication pouch picked up from the storage location to the discharge chute. As an alternative or in addition to the discharge chute, e.g. a conveyor may be placed underneath the collector, such as a belt or slat conveyor, which is configured to transfer the dose medication pouches collected from the storage locations to a dispensing point alike the dispensing station 18 of alike discharge chute alike shown in FIGS. 1 to 3.

As mentioned above, the number of modules and thereby the size of the drug cabinet belonging to the apparatus according to the invention may vary as well. In an embodiment significantly larger than the embodiment of FIGS. 1 to 3, the number of collectors may also be two or more instead of one, for example in such a way that the collectors move past each other in a staggered way, or in such a way that the collectors are placed next to each other or on different levels in the vertical direction, whereby each collector is responsible for the collecting of dose medication pouches from storage locations in a given area. Furthermore, in an embodiment, two or more dispensing stations may be provided instead of one dispensing station. For example, in an embodiment with two collectors next to each other, a dispensing station may be provided for each collector on the side wall of the drug cabinet of the apparatus in such a way that the collector of the left side takes the dose medication pouches collected from storage locations on the left half of the drug cabinet to the dispensing station on the left side, and the collector on the right side takes the dose medication pouches collected from storage locations on the right half of the drug cabinet to the dispensing station on the right side. Consequently, the apparatus according to the invention is not limited to the example embodiments presented above, but it may vary within the scope of the appended claims.

1. An apparatus for handling and dispensing dose medication pouches, wherein the dose medication pouches are stored in cassettes for dose medication pouches, and the apparatus comprises:
a control system for administration of data relating to storage location, drugs and use-by dates of the dose medication pouches, as well as for controlling functions of the apparatus;
a drug cabinet comprising storage locations provided for the cassettes of dose medication pouches and extending through the drug cabinet from a filling side to a dispensing side;
indicators on the filling side of the drug cabinet, for controlling the filling of the cassettes of dose medication pouches;
a collector on the dispensing side of the drug cabinet, configured to collect the dose medication pouches from the cassettes of dose medication pouches placed in the storage locations, according to the data relating to the storage location, the drugs and the use-by dates;
a dispensing station, to which the collector is configured to transfer the dose medication pouches retrieved from the cassettes of dose medication pouches.

2. The apparatus according to claim 1, further comprising a labelling device and/or a printing device for marking the collected dose medication pouches with the desired administration data.

3. The apparatus according to claim 1, wherein the dose medication pouches to be handled and dispensed are arranged in a long band of dose medication pouches in the cassettes of dose medication pouches, and wherein the apparatus comprises a cutting device for detaching the dose medication pouches to be taken from the cassettes of dose medication pouches and to be transferred to the dispensing station, from the other dose medication pouches in the band of dose medication pouches.

4. The apparatus according to claim 1, wherein the collector is configured to move in the horizontal and vertical directions along guide tracks on the dispensing side of the drug cabinet.

5. The apparatus according to claim 4, further comprising a manipulator mounted to be movable in at least one direction in relation to the collector and swiveling in relation to at least one axis, comprising at least one pincher roll or a similar rotatable pulling means for pulling out the band of dose medication pouches from the cassette of dose medication pouches.

6. The apparatus according to claim 5, wherein the manipulator comprises a transfer grip, into which, the manipulator receives the dose medication pouch pulled out of the cassette of dose medication pouches.

7. The apparatus according to claim 5, wherein the manipulator comprises a positioning sensor connected to the control system controlling the apparatus, for controlling the pulling out of the band of dose medication pouches.

8. The apparatus according to claim 4, wherein the collector comprises an industrial robot with an arm, the manipulator for dose medication pouches being provided at the end of the arm.

9. The apparatus according to claim 1, wherein the dispensing station comprises a discharge chute extending from the range of movement of the collector to the outside of walls of the drug cabinet, the dose medication pouches collected from the storage locations of the drug cabinet being guided along the discharge chute to be within reach of a user of the apparatus.

10. The apparatus according to claim 1, further comprising doors on the filling side of the drug cabinet, openable and lockable by electric locks.

11. The apparatus according to claim 10, wherein the doors are made of glass or another transparent sheet material.

12. The apparatus according to claim 1, wherein the drug cabinet is formed of two or more modules mounted next to each other.

13. The apparatus according to claim 12, wherein one door is provided for each module.

14. The apparatus according to claim 12, wherein each module comprises a rack with two or more shelves whose width is determined so that two or more cassettes of dose medication pouches are placeable next to each other on the shelves.

15. The apparatus according to claim 1, wherein the apparatus comprises two or more collectors.

16. The apparatus according to claim 1, wherein the apparatus comprises two or more dispensing stations.
